(19) United States
(12) Patent Application Publication
(10) Pub. No.: US 2004/0147940 A1
(43) Pub. Date: Jul. 29, 2004

(54) System and method to subdurally locate a catheter or lead

(75) Inventor: Christopher A. Crawford, Grapevine, TX (US)

Correspondence Address: Koestner Bertani, LLP, P.O. Box 26780, Austin, TX, 78755, US

(73) Assignee: Advanced Neuromodulation Systems, Inc., Plano, TX

(21) Appl. No.: 10/759,793

(22) Filed: Jan. 16, 2004

Related U.S. Application Data
(60) Provisional application No. 60/440,456, filed on Jan. 16, 2003.

(51) Int. Cl. A61B 19/00

(52) U.S. Cl. 606/129

(57) ABSTRACT

A tunneling tool forms a subdural pathway between a first incision and a second incision. This tool includes a shaft having a tunneling tip located at one end of the shaft. The tunneling tip is inserted through the first incision to tunnel the subdural pathway between the first incision and the second incision. A handle at the opposite end of the shaft guides the tunneling tip from the first incision to the second incision. A tapered slot in the shaft has a wide opening located opposite the tunneling tip and tapered sides that taper inwardly towards the tunneling tip. The wide opening receives an implantable line that the tapered sides secure to the tunneling tool. This allows the tunneling tool to be withdrawn along the subdural pathway to locate the line along the subdural pathway.

### RELATED APPLICATIONS

This application claims the benefit of priority under 35 USC 119(e) to U.S. Provisional Patent Application No. 60/440,456 entitled “SYSTEM AND METHOD TO SUB-DURALLY LOCATE A CATHETER OR LEAD”, filed on Jan. 16, 2003, and is incorporated herein by reference in its entirety.

### TECHNICAL FIELD OF INVENTION

This invention relates to a tool for implanting a catheter, lead or their extensions. More specifically, this invention relates to a tunneling tool to draw a catheter, lead or extension through subdural tissue.

### BACKGROUND OF INVENTION

Applications involving local drug delivery, tissue drainage, and Neuromodulation often employ implanted lines such as catheters or lead systems. Implantable catheters deliver therapeutic fluids for pain management, muscle disorder treatment, hormone and insulin regulation, and brain disorder treatments. Additionally, catheters drain fluids from wounds, ventricles in the brain, and other regions. Implantable leads deliver electrical signals to neuromodulate nerves to manage pain and treat disorders.

In such applications, the catheter, lead or extension thereof extends between the treatment area and the pump, signal generator, or depositing region. In so doing, the catheter or lead passes through various tissues to the terminal region. Often, this includes passing under the epidermis about the body to the terminal region.

The distal end of a lead system used to neuromodulate nerve tissues, located near the spinal cord or beneath the epidural layer of the spinal membranes, treats chronic pain and/or muscular disorders. The lead extends from the spinal region to an implantable pump or signal generator, typically placed under the epidermis on the side, near the abdomen, or lower back of the patient. The lead is surgically located near the spinal cord. Then, the lead is drawn through the tissues or under the skin to the location of the implantable pump or generator. Drawing the catheter or lead through the tissue or under the skin allows the number and size of incisions to be reduced. The reduced number and size of incisions facilitates the patient's recovery and reduces the chances for infection.

Various methods have been used to draw the catheter or lead through tissue. However, these methods typically require extensive manipulation of the end of the catheter or lead. Moreover, these methods often fail, forcing the patient to undergo surgery to repeat the tunneling procedure. One such method inserts a barbed gripping tool into the lumen of the catheter. First, the surgical team tunnels the tool through the tissue from the terminal region to a point close to the treatment region. The barbed end of the tool is inserted into the lumen of the catheter, gripping the internal surface. Then the surgical team draws the tool back through the tissue, pulling the catheter with the tool. However, the barbs often loosen or tear from the interior surface of the catheter, forcing surgical teams to repeat the process. Additionally, this process can damage the implantable line.

Another method sutures the catheter to the end of a tunneling tool. Such a method requires even more extensive manipulation of the catheter and therefore more time in surgery. As such, many typical tunneling tools and methods suffer from deficiencies in efficient tunneling of catheters and leads.

### SUMMARY OF INVENTION

The present invention provides a tunneling tool that substantially eliminates or reduces disadvantages and problems associated with previously developed catheter tunneling tools and methods that employ these tools. More specifically, the present invention provides a tunneling tool to place catheters or leads, collectively referred to herein as implantable lines, within tissues. The tunneling tool includes a thin shaft or rod. The distal end has a handle located at one end of the shaft. On the opposite end of the shaft, the tunneling end pushes tissue and fat out of the way to create the subdural pathway. A tapered slot, located near the proximal end, extends through the shaft. The implantable lines can be threaded through the slot. The slots taper pinches the implantable line to secure the implantable line while the tunneling tool is withdrawn.

In one embodiment, the shaft is an elongated member with a circular cross-section. Alternatively, a solid wire forms the shaft. In either case, the shaft narrows towards the tunneling tip. The shaft and tool may be made from a material that is bendable to hold a desired shape. In one embodiment, the handle connects to the distal end of the shaft with a coupling. The coupling may act as a stopping mechanism for a tube, which may fit on the outside of the shaft.

When tunneling, the surgical team bends the shaft to a desired shape and then guides the shaft through the tissue using the handle. Once the proximal end of the tool is extended to the treatment region and is accessible to the surgical team, the surgical team threads the implantable line through the tapered slot. The slot pinches the implantable line with its tapered sides. The surgical team then withdraws the tunneling tool back through the tissue with the implantable line.

A tube may be used with the tunneling tool. In this instance, the tube may remains in place within the tissue while the tunneling tool is withdrawn through the tissue. The implantable line is not coupled to the tunneling tool while the tool is withdrawn. The tube keeps the path of the tunneling tool open to thread or string the implantable line back through the subdural pathway. The tube reduces the friction between the implantable line and the surrounding tissue. Once in place, the surgical team may remove the tube.

These implantable lines facilitate the delivery of medications or electrical signals that neuromodulate nerve tissues. These tissues include, but are not limited to, the spinal column, brain, and other regions and organs. Additionally, this tool may be used to implant catheters that drain fluids or other applications known to those skilled in the art.

Another embodiment provides a method to implant implantable lines. The method involves tunneling with a tunneling tool through a tissue, from a terminal region to a treatment region. The tunneling tool has a handle located at a distal end and couples to a shaft. A tapered slot located near the proximate end tapers towards the proximate tunneling end. The tunneling tool extends to the treatment region. A receiving end of the implantable line is pinched within the tapered slot once the tunneling tool has been extended. This involves first threading the implantable line through the slot and wedging the implantable line into the taper. The tunneling tool and the receiving end of the implantable line are then withdrawn through the tissue. This strings the implantable line along the tunneled path.

As such, a tunneling tool and method for its use, and method for its manufacture are described herein. Other aspects, advantages and novel features of the present invention will become apparent from the detailed description of the invention when considered in conjunction with the accompanying drawings.

### BRIEF DESCRIPTION OF THE DRAWINGS

For a more complete understanding of the present invention and advantages thereof, reference is now made to the following description taken in conjunction with the accompanying drawings in which like reference numbers indicate like features and wherein:

FIG. 1 is a pictorial of one use of the present invention;

FIG. 2 is a schematic diagram of a catheter directed through a tapered slot in accordance with the present invention;

FIGS. 3A, 3B and 3C are schematic diagrams depicting one embodiment of the present invention;

FIG. 4 is a schematic diagram depicting one embodiment of the present invention;

FIG. 5 is a schematic diagram depicting one embodiment of the present invention;

FIG. 6 is a block flow diagram that describes the implantation of a implantable line in accordance with one embodiment; and

FIG. 7 depicts a neuromodulation therapy system wherein the implantable leads are placed in accordance with the present invention.

### DETAILED DESCRIPTION OF THE INVENTION

Preferred embodiments of the present invention are illustrated in the FIGUREs, like numerals being used to refer to like and corresponding parts of the various drawings.

The present invention provides a tunneling tool to place implantable lines, catheters or leads, subdurally. The tunneling tool includes a thin shaft or rod. The distal end has a handle located at one end of the shaft, while the opposite or proximal end has a tunneling tip. A tapered slot, located near the proximal end, extends through the shaft. The implantable line can be threaded through the slot wherein slot's taper pinches the line to secure line while the tunneling tool is withdrawn.

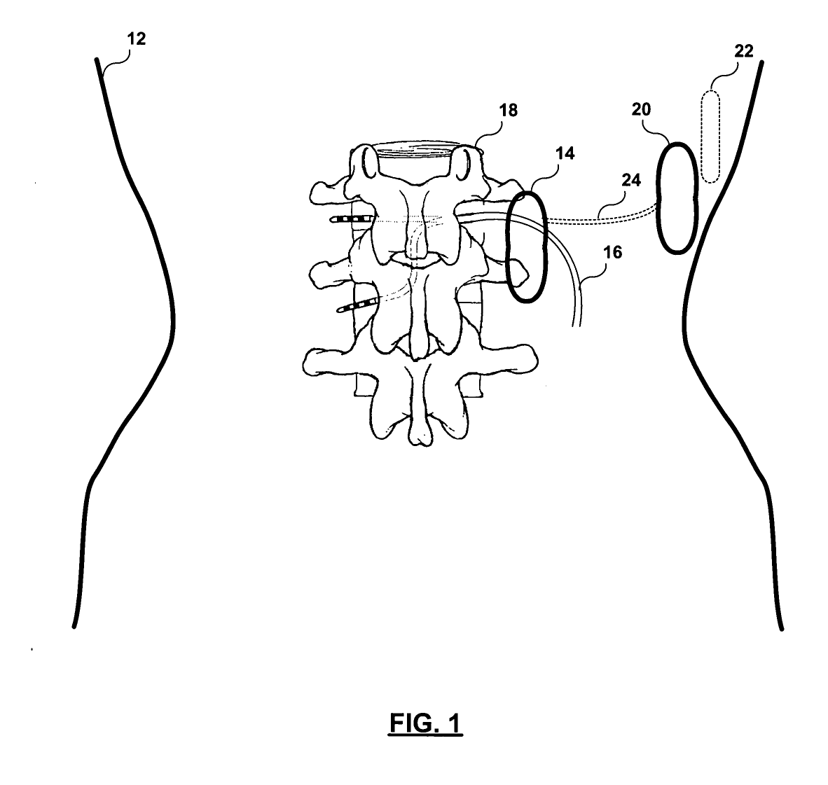

FIG. 1 depicts one embodiment of an implantable system to deliver medications or manage pain. In patient 12, an incision 14 provides access to the treatment region or spine 18. A surgical team inserts implantable line 16 into the epidural space in the spine. The implantable line extends out through incision 14. Typically, an implantable infusion pump or impulse generator 22 is placed subdurally in the abdominal region. In this instance, incision 20 is made at the terminal region and path 24 is tunneled under the skin between the incisions. Implantable line 16 is drawn back across the tunnel 24 to the location of the implantable therapeutic device 22. This device may be an implantable infusion system, implantable pulse generator or other like device known to those skilled in the art.

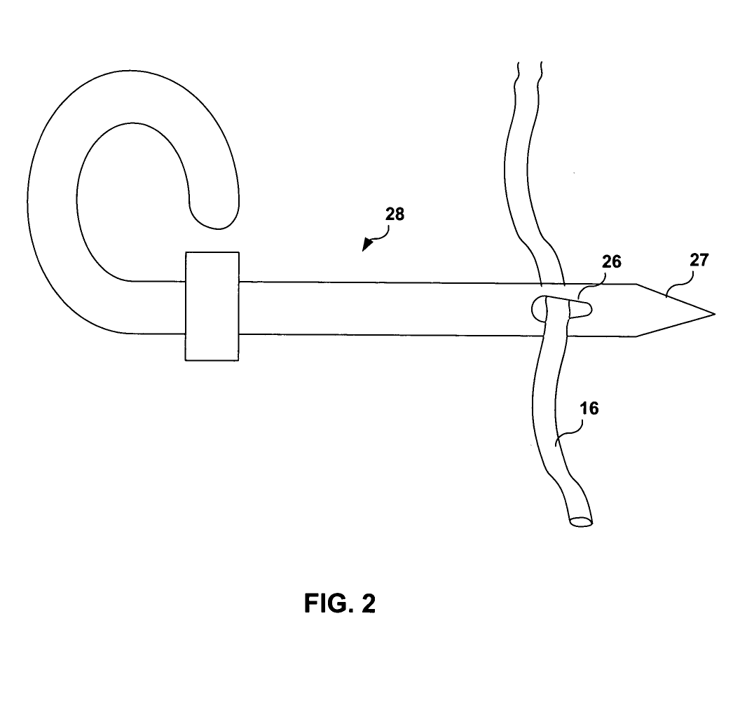

FIG. 2 is a schematic diagram of implantable line 16 directed through tapered slot 26 located near proximate end 27 of tunneling tool 28. Implantable line 16 is held in place by tapered slot 26. Tunneling end 27 pushes tissue and fat out of the way while the tool tunnels. Pushing these tissues and fats aside avoids the risk of cutting muscles, organs or other like structures. The secured implantable line 16 is withdrawn along the tunneling path 24 of FIG. 1. One will recognize that these procedures also are applicable to tunnel any subdural path for an implantable line.

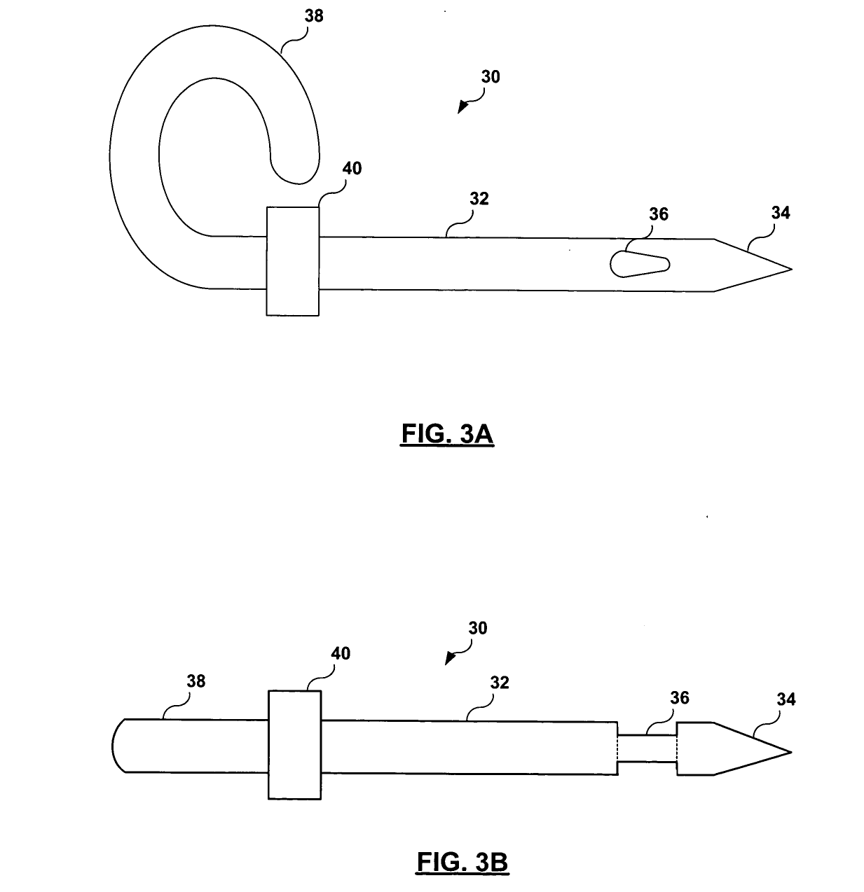

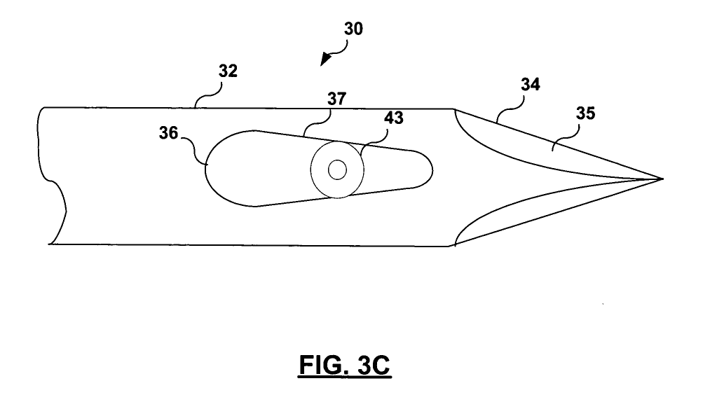

FIGS. 3A, 3B and 3C depict one embodiment of the present invention. Here, tunneling tool 30 includes a shaft 32 that terminates in tunneling end 34. Near tunneling end 34, slot 36 extends through the shaft 32. Slot 36 at its widest point is wider than the catheter, lead or extension thereof. However, slot 36 tapers towards tunneling end 34 to an opening narrower than the catheter. FIG. 3C shows catheter 43 being inserted into hole 36 and drawn towards the tunneling end 34. This allows the tapered walls 37 of the slot to pinch catheter 42, holding catheter 42 securely while tool 30 is withdrawn.

Handle 38 may be formed by bending shaft 32, or coupled to shaft 32 with connector 40. Connector 40 may be crimped, bonded or otherwise mechanically coupled to the shaft and handle as known to those skilled in the art. Shaft 32, handle 38 and connector 40 may be made from various materials including, but not limited to, surgical steel, stainless steel, 304 stainless steel, 303 stainless steel, various metals, various alloys, various composites, various plastics, or other compatible material known to those skilled in the art. Slot 36 may be formed through various means, including milling, etching, laser cutting, EDM, and other means known to those skilled in the art.

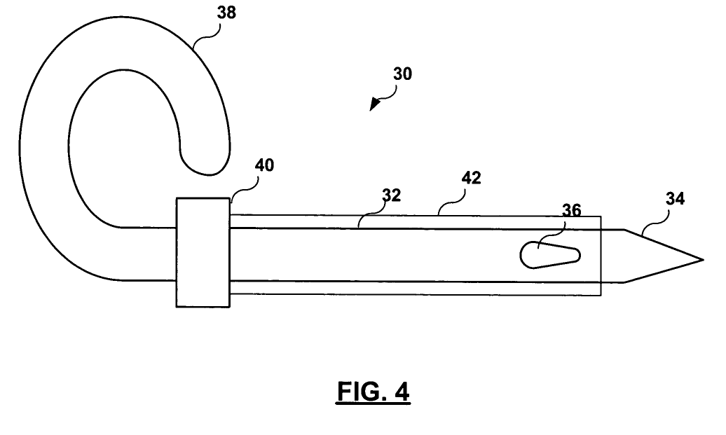

FIG. 4 depicts another embodiment of the present invention. In this embodiment, shaft 32 has a tapered slot 36 near a tunneling end 34. As in FIGS. 3A, 3B and 3C, slot 36 tapers towards the tunneling end 34 and is wider opposite the tunneling end. Handle 38 mechanically couples to shaft 32 with a connector 40 on the distal end. Tube 42 concentrically surrounds the shaft 32. Connector 40 secures tube 42 during insertion, tunneling and extraction. Tunneling tool 30 is guided subdurally between an implanting or terminal region to a location or incision near the treatment area. The catheter may be inserted into tube 42 and threaded through the tube to the implanting or terminal region.

The tube may remain in place to protect the implantable line or be removed after the implantable line has been located and prior to affixing the catheter to the therapeutic device. In this manner, tube 42 protects the implantable line along the tunneled path. Tube 42 may be made of various materials including plastics, surgical steel, stainless steel, various metals and alloys, and various combinations of these, among others.

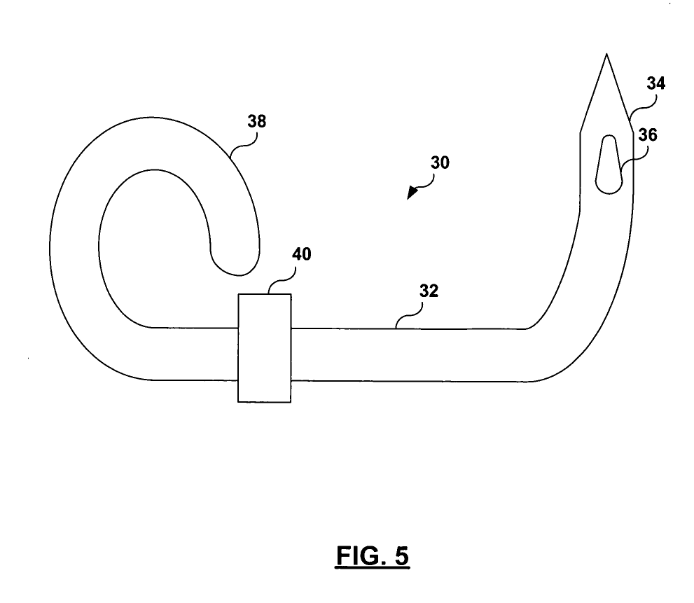

In FIG. 5, shaft 32 is bent. This shape, when shaft 32 is made of a material that maintains its shape once bent, allows the tool to be guided around muscles, bones, and organs, or other internal structures. For example, a surgical team may bend shaft 32 to conform to the shape around the torso. In this manner, the tunneling tool may be guided subdurally about the abdominal muscles and back structures.

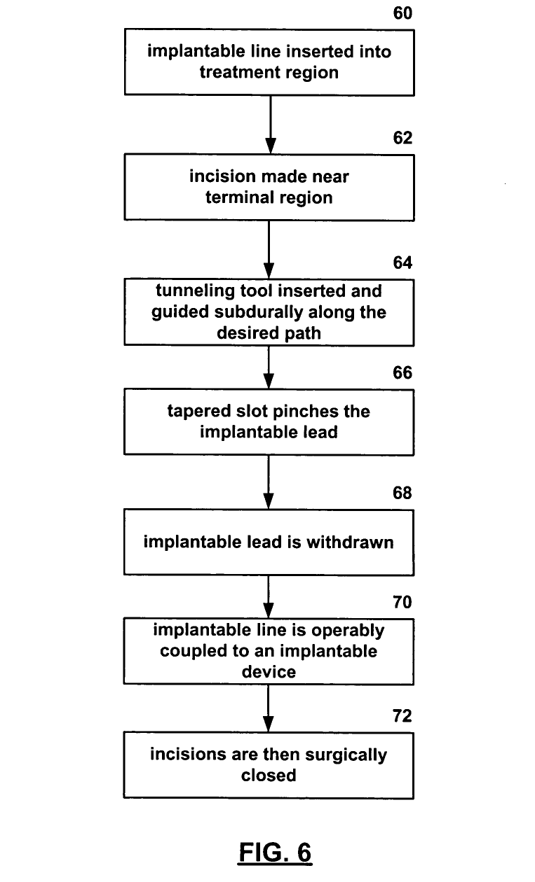

FIG. 6 is a process flow diagram depicting one embodiment of employing the tunneling tool. The implantable line is first inserted in a treatment region in step 60. For example, a lead system may be placed intra-spinally for treatment of chronic pain or muscle spasms, among others. In other embodiments, the implantable line may be placed near regions of the brain, various organs, and near regions of absorption. Alternately, the implantable line may be placed near the terminal region and drawn back subdurally to the treatment site.

At step 62, an incision is made near the terminal region. The surgical team then prepares the tunneling tool to tunnel subdurally. For example, the tool may be bent to conform to the tissue structures or desired tunneling path. In addition, a tube operably coupled to the shaft may be inserted over the shaft of the tunneling tool.

In step 64, the tunneling tool is inserted and guided subdurally along the desired path from the implant incision to the implantable line insertion incision. The surgical team then accesses the tunneling tip of the tunneling tool. Specifically, the surgical team accesses the tapered slot which now extends from the implantable line's insertion incision. The surgical team threads one end of the implantable line into the tapered slot. Then, the tapered slot in step 66 pinches the implantable line. This secures the implantable line to the tunneling tool. The implantable line is then drawn back through the tunnel created in step 64 in step 68. This involves drawing the catheter and tunneling tool back through the tunnel created by the insertion of the tunneling tool.

The implantable line is operably coupled to an implantable device such as an infusion pump, a terminating connector, or signal generator as seen in step 70. In the case of the implantable infusion pump or generator, the pump or generator is implanted subdurally proximate to the implant incision. The incisions are then surgically closed at step 72. As one will appreciate, however, various embodiments of the method and applications for the tunneling tool are possible. The tool may be used to draw catheters, lead or extensions thereof from the terminal location to the treatment location.

The tunneling tool is manufactured to have a tapered slot in the shaft as seen in the FIGUREs. The slot tapers towards the tunneling tip of the shaft. At the wider portion of the slot, the slot is wider than a catheter, lead or extensions thereof. The slot may be created by milling, etching, laser cutting, or other like means known to those skilled in the art. Alternatively, the slot may be formed by milling a small portion of the hole and burning or etching material from the hole with the use of a hot wire or EDM method. However, any methods known to those skilled in the art may be employed. The tunneling tip end of the shaft near the slot pushes tissue aside to facilitate tunneling.

On the distal end of the shaft a handle is operably coupled to or bent from the shaft. The coupling may use various methods including welding, adhering, centering, screwing, bonding or other like methods known to those skilled in the art. An intermediary connector or bushing may also be used to facilitate this coupling. In one embodiment, the intermediary connector or bushing has a diameter greater than that of the shaft and acts as a stop for a tube.

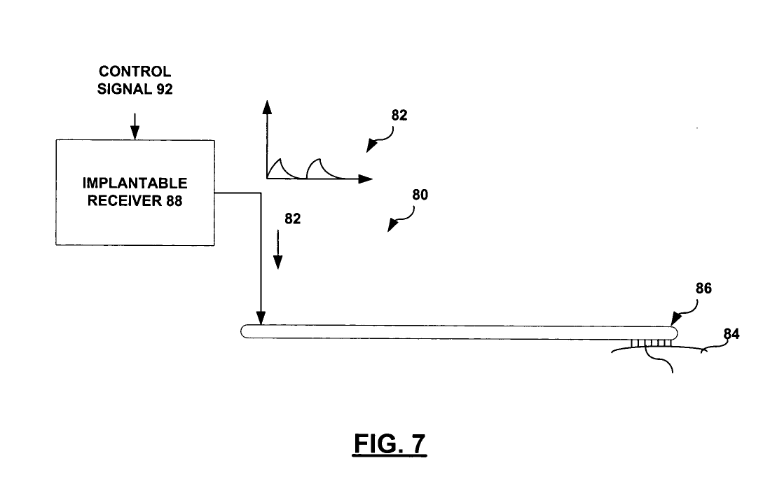

FIG. 7 shows a neuromodulation system or tissue stimulator system 80 used to apply electrical stimulation in the form of electrical signal 82 to tissue 84. The implantable line may be placed using the tunneling tool of the present invention. Implantable line 86 is located proximate to tissue 84 and electrically and operably coupled to a controller or processor. Implantable receiver 88 may be physically or logically located within a controller or processor.

Implantable receiver 88 receives a control signal 92 from the processor. Upon receiving control signal 92, receiver (or implantable pulse generator) 88 produces electrical stimulation signal 82 for implantable stimulation lead 86 that travels through an inert housing or header, which actually receives the stimulation lead. This electrical stimulation signal may take the form of a series of generator electrical pulses.

Controller or processor may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. Memory operably coupled to the processor may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the controller or processor implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory stores, and the controller or processor 90 executes operational instructions to the functions of the neuromodulation system.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

Although the present invention is described in detail, it should be understood that various changes, substitutions and alterations can be made hereto without departing from the spirit and scope of the invention as described by the appended claims.

What is claimed is:

1. A tunneling tool, to form a subdural pathway between a first incision and a second incision comprising:
a shaft having a first end and second end located at opposite ends of the shaft;
a tunneling tip located at the first end of the shaft, wherein the tunneling tip is inserted through the first incision to tunnel the subdural pathway between the first incision and the second incision;
a handle at the second end of the shaft operable to guide the tunneling tip from the first incision to the second incision; and
a tapered slot in the shaft proximate to the tunneling tip, wherein the tapered slot further comprises:
a wide opening at located opposite from the tunneling tip; and
tapered sides that taper inwardly towards the tunneling tip, wherein the wide opening receives a line and the tapered sides secure the line to the tunneling tool, and wherein the tunneling tool is withdrawn along the subdural pathway to locate the line along the subdural pathway.

2. The tunneling tool of claim 1, wherein the handle comprises a bent portion of the shaft.

3. The tunneling tool of claim 1, further comprising a connector operable to couple the handle to the shaft.

4. The tunneling tool of claim 1, further comprising a tube concentrically counted about the shaft, wherein the connector acts as a stop for the tube.

5. The tunneling tool of claim 4, wherein the tube remains located along the subdural pathway to protect the catheter, when the tunneling tool is withdrawn from the subdural pathway.

6. The tunneling tool of claim 1, wherein:
the first incision is located proximate to a terminal region; and
the second incision is located proximate to a treatment region.

7. The tunneling tool of claim 1, wherein:
the second incision is located proximate to a terminal region; and
the first incision is located proximate to a treatment region.

8. The tunneling tool of claim 7, wherein the line comprises a catheter operable to deliver medication to the treatment region.

9. The tunneling tool of claim 7, wherein the line comprises a catheter operable to drain the treatment region.

10. The tunneling tool of claim 7, wherein the line comprises a lead operable to deliver electrical signals to neuromodulate tissues from a signal generator and to electrodes proximate to the treatment region.

11. The tunneling tool of claim 10, wherein the lead is implanted proximate to neural tissue proximate to the treatment region and the signal generator is implanted in the terminal region.

12. A method to form a subdural pathway between a first incision and a second incision for an implantable line, comprising:
inserting a tunneling tip of a tunneling tool into a first incision;
guiding the tunneling tool subdurally from the first incision to the second incision to form the subdural pathway;
securing the line to the tunneling tool with a tapered slot proximate to the tunneling tip that pinches the line; and
backing the tunneling tool out from the first incision along the subdural pathway to lay the implantable line along the subdural pathway.

13. The method of claim 12, wherein the tunneling tool comprises:
a shaft having a first end and second end located at opposite ends of the shaft, wherein the tunneling tip is located at the first end of the shaft;
a handle at the second end of the shaft operable to guide the tunneling tip from the first incision to the second incision; and
wherein the tapered slot further comprises:
a wide opening at located opposite from the tunneling tip; and
tapered sides that taper inwardly towards the tunneling tip, wherein the wide opening receives the implantable line and the tapered sides secure the line to the tunneling tool.

14. The method of claim 13, wherein the handle comprises a bent portion of the shaft.

15. The method of claim 13, wherein the tunneling tool further comprises a tube concentrically mounted about the shaft, wherein the tube remains located along the subdural pathway to protect the implantable line.

16. The method of claim 12, wherein:
the first incision is located proximate to a terminal region; and
the second incision is located proximate to a treatment region.

17. The method of claim 16, wherein the implantable line comprises a catheter operable to deliver medication to the treatment region.

18. The method of claim 16, wherein the implantable line comprises a catheter operable to drain the treatment region.

19. The method of claim 16, wherein the implantable line comprises a lead operable to deliver electrical signals to neuromodulate tissues from a signal generator and to electrodes proximate to the treatment region.

20. The method of claim 19, wherein the lead is implanted proximate to neural tissue proximate to the treatment region and the signal generator is implanted in the terminal region.

21. A neuromodulation therapy system comprising:
at least one implantable stimulation lead to deliver electrical energy to proximately positioned tissue, wherein the implantable stimulation lead is located subdurally with a tunneling tool; and
an implantable pulse generator coupled to the at least one stimulation lead, wherein the implantable pulse generator is operable to deliver electrical energy to the at least one stimulation lead in response to at least one control signal.

22. The neuromodulation therapy system of claim 21, wherein the tunneling tool comprises:
a shaft having a first end and second end located at opposite ends of the shaft;
a tunneling tip located at the first end of the shaft, wherein the tunneling tip is inserted through the first incision to tunnel the subdural pathway between the first incision and the second incision;
a handle at the second end of the shaft operable to guide the tunneling tip from the first incision to the second incision; and
a tapered slot in the shaft proximate to the tunneling tip, wherein the tapered slot further comprises:
a wide opening at located opposite from the tunneling tip; and
tapered sides that taper inwardly towards the tunneling tip, wherein the wide opening receives the at least one implantable stimulation lead and the tapered sides secure the at least one implantable stimulation lead to the tunneling tool, and wherein the tunneling tool is withdrawn along the subdural pathway to locate the at least one implantable stimulation lead along the subdural pathway.

23. The neuromodulation therapy system of claim 22, wherein the handle comprises a bent portion of the shaft.

24. The neuromodulation therapy system of claim 22, further comprising a connector operable to couple the handle to the shaft.

25. The neuromodulation therapy system of claim 22, further comprising a tube concentrically counted about the shaft, wherein the connector acts as a stop for the tube.

26. The neuromodulation therapy system of claim 25, wherein the tube remains located along the subdural pathway to protect the catheter, when the tunneling tool is withdrawn from the subdural pathway.

27. The neuromodulation therapy system of claim 22, wherein:
the first incision is located proximate to a terminal region; and
the second incision is located proximate to a treatment region.
